(19) United States
(12) Patent Application Publication
(10) Pub. No.: US 2023/0310084 A1
(43) Pub. Date: Oct. 5, 2023

(54) SPINAL SURGERY PLANNING SOFTWARE

(71) Applicant: Formus Labs Limited, Grafton (NZ)

(72) Inventors: Ju ZHANG, Auckland (NZ); Thor Franciscus BESIER, Auckland (NZ)

(21) Appl. No.: 18/329,613

(22) Filed: Jun. 6, 2023

Related U.S. Application Data
(63) Continuation of application No. PCT/NZ2021/050218, filed on Dec. 9, 2021.

(30) Foreign Application Priority Data
Dec. 9, 2020 (NZ) 770600

(51) Int. Cl. A61B 34/10

(52) U.S. Cl. CPC A61B 34/10; A61B 2034/102; A61B 2034/107; A61B 2034/105

(57) ABSTRACT

A method of determining the effect of a surgical procedure on a subject's spine. The method includes receiving geometric parameters of the subject's spine, constructing a virtual model of the subject's spine using the geometric parameters, and determining the effect of the surgical procedure on the subject's 5 spine using the virtual model. The virtual model includes node units connected by connector units, where the connector units include virtual springs. The method can also include optimising parameters of a surgical procedure by reducing the error between a target spine and the spine model modified by the surgical procedure. A computer program for performing the method is also described.

### CROSS-REFERENCE TO RELATED APPLICATIONS

This is a bypass continuation of International PCT Application No. PCT/NZ2021/050218, filed on Dec. 9, 2021, which claims priority to New Zealand Patent Application No. 770600, filed on Dec. 9, 2021, which are incorporated by reference herein in their entirety.

### FIELD

This invention relates to spinal surgery planning software.

### BACKGROUND

Spinal procedures like disc fusion, scoliosis correction and spondylolisthesis correction are commonly performed to correct spinal defects, degeneration, or malformations. Surgical procedures typically include implantation of a spinal implant and resection (removal) of a portion of vertebral bone to correct spinal posture and restore function.

Various types of spinal implants are available, which can be placed in a variety of positions, orientations, size, shape to achieve desired spinal posture and vertebral spacing. Portions of bone of various sizes, shapes and locations can also be resected to allow additional spinal curvature to be achieved. These options allow a surgeon to tailor a procedure to a given patient based on their condition and a target clinical outcome.

However, there is much variability between different patients' anatomies, conditions, and spinal function. This variability combined with the variations possible in implant selection and placement results in a wide range of possible outcomes for the patient, some desirable, some not. Therefore, it can be difficult for a surgeon to know in advance of surgery, or even during surgery, what effect a procedure will have on a particular patient and to plan a procedure accordingly without predictive tools.

Predictive pre-operative planning software has the potential to help the surgeon determine the optimal procedure for a particular patient. Ideally the software should predict the patient's post-operative spinal posture and function from a given set of procedure parameters (e.g. implant selection, placement, resection). This capability would allow a surgeon to explore different procedure parameters to find the set that gives the best surgical outcome in his or her opinion. Alternatively, given some surgical outcome targets, the software could in theory automatically find the set of procedure parameters that best achieves those targets.

However, the spine is a complex system consisting of a multitude of passive and active elements. Some predictive pre-operative planning methods could be either prohibitively time consuming to set up and run in a clinical setting, or limited to very simple planning tasks on a small set of implants.

It would be advantageous to provide simplified computational models of the spine that were more feasible in a clinical setting. It would also be advantageous to provide models that could incorporate patient-specific material properties and that had the ability to simulate bone resection.

### SUMMARY

According to one example embodiment there is provided a method of determining the effect of a surgical procedure on a subject's spine, the method including: receiving geometric data of the subject's spine; constructing a virtual model of at least part of the subject's spine using the geometric data, the virtual model including a series of node units representing vertebrae and a plurality of connector units, each connector unit including one or more virtual springs configured to model the coupling between adjacent node units; and determining the effect of a surgical procedure on the subject's spine using the virtual model.

According to another example there is provided a computer program for determining the effect of a surgical procedure on a subject's spine, the computer program including computer-readable instructions for causing a computer to: upon receiving geometric data of the subject's spine, construct a virtual model of at least part of the subject's spine using the geometric data, the virtual model including a series of node units representing vertebrae and a plurality of connector units, each connector unit including one or more virtual springs configured to model the coupling between adjacent node units; and using the virtual model, determine the effect of a surgical procedure on the subject's spine.

According to another example there is provided a non-transitory computer-readable medium having stored thereon computer-readable instructions for causing a computer to: upon receiving geometric data of the subject's spine, construct a virtual model of at least part of the subject's spine using the geometric data, the virtual model including a series of node units representing vertebrae and a plurality of connector units, each connector unit including one or more virtual springs configured to model the coupling between adjacent node units; and using the virtual model, determine the effect of a surgical procedure on the subject's spine.

According to another example there is provided a method of determining the effect of a surgical procedure on a subject's spine, the method including: receiving geometric data of the subject's spine; constructing a virtual model of at least part of the subject's spine using the geometric data, the virtual model including a series of node units representing vertebrae and a plurality of connector units, each connector unit including one or more virtual springs configured to model the coupling between adjacent node units, wherein the coupling been adjacent node units represents an intervertebral disc; and determining the effect of a surgical procedure on the subject's spine using the virtual model.

### BRIEF DESCRIPTION OF THE DRAWINGS

The accompanying drawings which are incorporated in and constitute part of the specification illustrate embodiments of the invention and, together with the general description of the invention given above, and the detailed description of embodiments given below, serve to explain the principles of the invention.

FIG. 1 is a flow chart illustrating a method of determining the effect of a surgical procedure;

FIG. 2 is a model of a spine according to one embodiment;

FIG. 3 is a flow chart illustrating a method of constructing a model of a spine according to one embodiment;

FIG. 4 is a flow chart illustrating a method of constructing a model of a spine according to one embodiment;

FIG. 5 is a flow chart illustrating a method of optimising parameters of a surgical procedure according to one embodiment;

FIG. 6 is a front view of a model of a spine according to another embodiment;

FIG. 7 is a side view of the model of FIG. 6;

FIG. 8 is a model of part of a spine according to another embodiment;

FIG. 9 is a model of part of a spine according to another embodiment; and

FIG. 10 is a model of a spine augmented with a surgical implant according to one embodiment.

### DETAILED DESCRIPTION

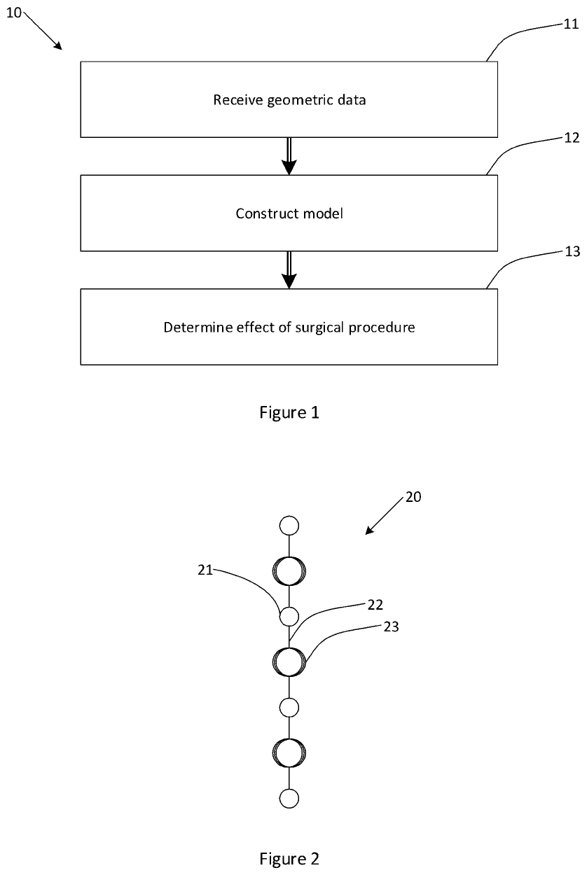

FIG. 1 shows a method 10 of determining the effect of a surgical procedure on a subject's spine. This method may improve the ability of a surgeon to predict what effect a surgical procedure will have on the subject, taking into account subject-specific anatomical features. The method may also assist a surgeon in determining optimal parameters to achieve a target clinical outcome for the subject.

Initially, geometric data of the subject's spine is received 11. The geometric data can be obtained from one or more radiographic images, computerised tomography (CT) scans, magnetic resonance imaging (MRI), body surface scans or other suitable techniques and combinations of these techniques. The radiographic images can be X-ray images, for example standing weight-bearing images. The X-ray images can be two or more X-ray images taken at different angles. These X-ray images can be combined using stereophotogrammetry to produce three-dimensional image data.

In one example, the subject's spine is imaged to determine geometry of the subject's vertebrae, which can include the size, shape and position of the vertebrae in three dimensions. The geometry of the vertebrae can be determined from CT or MRI data using automatic image segmentation. Alternatively, the vertebrae could be manually identified and delineated in the CT or MRI data. The geometry of the vertebrae could also be determined from stereophotogrammetry of X-ray images. The geometry of the vertebrae could also be determined from the non-rigid registration of a 3D statistical shape model to a sparse set of features or landmarks in the X-ray, CT, or MRI data.

The geometric data of the subject's spine can be determined for multiple poses. This can involve having the subject assume multiple different poses during imaging of the spine. Alternatively, a deformable model of the subject's torso may be used to extrapolate from spine geometry in one pose to spine geometry in other poses. This could involve determining the 3D geometry of the vertebrae from CT or MRI data; taking an X-ray image of the subject in one pose; embedding the vertebrae in the deformable model, where the vertebrae have geometries based on the CT or MRI data and positions based on the X-ray image; obtaining body surface scans of the subject in a range of poses; and morphing the deformable model into the range of poses based on the body surface scans. The deformable model can be a statistical shape model containing the spine, soft tissue and skin surface. The body surface scans can be obtained using Microsoft Kinect or Intel RealSense camera systems or other suitable surface scan systems.

In addition to imaging, functional assessments may be used to assess the subject. These can involve measuring the subject using wearable sensors as they perform various movements such as flexion and extension, left-right bend, spinal twist and sit to stand. The wearable sensors could include, for example, inertial measurement units (IMUs), flexible capacitive sensors or dynamometers.

A virtual model of the subject's spine is then constructed 12 using the geometric data and optionally also the functional data. The model may also be based on muscle parameters such as shape, volume, type, density or mass; bone parameters such as shape, volume, density, mass or stiffness; or intervertebral disc parameters such as shape, volume, density, mass or stiffness. Taking these additional elements into account may improve the concordance between the model and the subject's spine and produce a more realistic simulation. It will be understood that only part of a subject's spine may be modelled in some cases and that references to a model of a subject's spine also cover models of a part of the subject's spine.

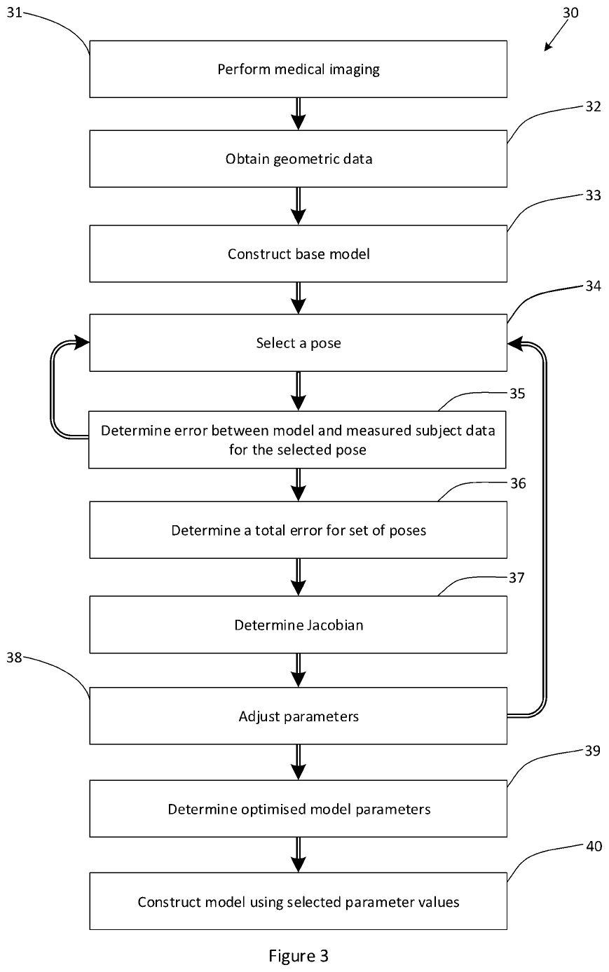

As will be described in more detail with reference to FIG. 3, the virtual model can be constructed by adjusting parameters of a base model of a spine to optimise the match between the virtual model and the subject's spine. Various suitable optimisation strategies and algorithms may be used. Various suitable metrics may be assessed to determine how well the model is optimised to the subject's spine.

Once the virtual model is constructed, the effect of a surgical procedure on the spine is determined 13. The virtual model is modified based on the surgical procedure to simulate the subject's spine as it would be after the procedure. As will be described in more detail with reference to FIG. 5, modifying the model can involve augmenting the virtual model with a node that represents an implant and/or modifying a node of the virtual model in a way that represents resection of part of a vertebra. The virtual model of the spine will respond to the modification in a way that simulates the response of the subject's spine to the surgical procedure. This response can then be assessed by a surgeon to decide if the surgical procedure is suitable for the subject or can be used to optimise parameters of the surgical procedure to achieve a suitable post-operative outcome for the subject. The response to the procedure can also be assessed intra-operatively to determine the effect of planned procedure components (e.g. planned implants) based on one or more other components (e.g. another implant) that have already been delivered. Similarly, parameters of planned procedure components (e.g. implants) can be optimised given the fixed parameters of one or more already delivered components (e.g. implants). Post-operatively, the model may be useful for predicting post-operative function based on the actual parameters of the delivered components (implants and/or resections).

FIG. 2 shows a simple virtual model 20 of a subject's spine. The model is made up of a series of node units 21 that are connected by connector units 22. Each connector unit includes a spring 23. The node units 21 can represent vertebrae and connector units 22 can represent the connections between vertebrae, such as intervertebral discs, ligaments and muscles. The nodes can be represented as spatially extended bodies to represent the size and shape of corresponding vertebrae. The model can include other elements such as masses, damping elements and additional springs to better simulate the subject's spine. Generally speaking, the model represents each vertebra as a node and the local mechanical environment of that vertebra as one or more elements connected to the node. The model may be considered a lumped parameter model, in which each major component of the real system is represented by one or a combination of basic elements such as springs, masses, dampers, impedances, force sources etc.

The inventors have found that such a model can adequately simulate a real spine and its response to surgical procedures while being computationally efficient. In particular, springs can capture key behaviour of intervertebral connections in a way that could be particularly difficult using other modelling techniques. A traditional approach to modelling a subject's anatomy would involve building as detailed a model as feasible, for example using finite element modelling or a detailed 3-dimensional beam model with many complex deformation modes. These other methods of simulating a spine may be very computationally intensive and require detailed knowledge of the physical properties of the spine and its environment and the distribution in space of these properties.

Also, springs may provide much simpler models of intervertebral connections than other models, such as beam models for example. Beam models may require many variables to define the beam parameters. Beams have finite thickness and undergo compression on one side and tension on the other during bending. Beam models may be described by a stiffness matrix with many coefficients relating shear forces and bending moments on a modelled object to deflections and rotations of the object. The coefficients account for various deformations including shear deformations. Beam deformations include complex deformations i.e. deformations in one dimension in response to applied forces in another dimension. These models are complex and computationally intensive to work with and can be extremely difficult to optimise due to the large number of parameters. Springs, in contrast, can be 1-dimensional with no inherent thickness. They do not have complex deformation modes like beams.

The deformation of the connections between nodes can be modelled by the springs alone. Each spring can have a single deformation mode in which it deforms in the same direction as an applied force. For a linear spring this is a linear deformation (extension or contraction) in a linear direction; for a torsion spring it is a torsional deformation about a single axis. This allows deformation to be modelled using only simple deformation modes without complex deformations like beam models.

In the example of FIG. 2, the springs 23 are torsion springs. In other examples, the springs can be linear springs. Each spring 23 can be connected to the centre of mass of a node 21 or, if the node is modelled as a spatially extended object, to a point on the surface of the nodes 21 or somewhere between the surface and the centre of mass. Each spring has a spring coefficient k which can be adjusted when optimising the model. For a torsion spring, the spring coefficient is a torsion coefficient; for a linear spring, the spring coefficient is a linear coefficient. Each spring may be a simple or constant-coefficient spring, in which the spring coefficient k is a constant, or it may be a variable-coefficient spring in which the coefficient k varies with displacement. The model can further include connector units that connect non-adjacent node units. These can span multiple node units to simulate muscles and ligaments.

FIG. 3 is a flow chart showing a detailed exemplary procedure 30 for constructing a model that is optimised to match a subject's spine. Initially, measurements are made on the subject to obtain measured subject data. This includes performing medical imaging 31 on the subject. In this example, this is performed for multiple poses of the subject. The images are then analysed to obtain geometric data 32 of the subject's spine. The geometric data can be measurements of various parameters including position, orientation, size and shape of vertebrae; position, size and shape of intervertebral discs. The geometric data can also be derived from two or more measurements, for example separation between two vertebrae (based on the differences in their positions), changes in the separation between two vertebrae between different poses, changes in the position or orientation of a vertebra between different poses, changes in an intervertebral disc between different poses etc. The position and orientation of the subject's sacrum and/or pelvis is also imaged. The geometric data can also be a combination of partial geometric data extracted directly from medical images and reconstructed measurements from statistical models. In this instance, the direct measurements, which may also include patient demographics and morphometric data, are provided as inputs to statistical models such as linear and non-linear regression models or statistical shape models, to reconstruct the remaining geometric data needed by the base model. For example, the geometry of every second vertebrae may be directly extracted from medical images while a statistical shape model of the spine uses that data to reconstruct the geometries of the other vertebrae. As another example, the geometry of the body surface is extracted from 3D body surface scans, and a statistical shape model of the body plus spine uses that data to reconstruct the spine geometry. As another example, geometric data in the form of landmark points are extracted from one or more X-ray images which are then used by a statistical shape model combined with stereophotogrammetry to reconstruct the 3D spinal geometry.

The geometric data are used to construct a base model in step 33. This can involve aligning a base of the model to the patient's sacrum or pelvis based on its determined location and orientation. The base provides a reference object for the rest of the model. The geometric properties of the node units and connector units are then defined based on the geometric parameters obtained from the imaging step. Next the units are assigned an initial set of mechanical parameters, e.g. spring coefficients, masses, damping factors etc. The initial mechanical parameters can be a generic set of parameters that are the same for any subject. Alternatively, the mechanical parameters of the base model can be determined from a statistical model taking into account factors such as the subject's demographic data, anthropometry, medical imaging data etc.

The procedure then moves to a numerical optimisation loop 34-38 that optimises parameters of a base model to simulate the subject's spine.

Firstly, a pose is selected 34, which for illustrative purposes is standing upright. The model (with the initial parameter values on the first iteration and the latest adjusted parameter values on subsequent iterations) is placed into this pose and a forward simulation of the model is performed. The model will adopt a posture that is a balance of the forces applied to it by the springs, force sources, mass etc. The posture of the model in this pose is then compared to measured data indicative of the posture of the subject in the same pose, which in this case can be geometric data specifying the positions of the subject's vertebrae when standing upright. The comparison is performed by measuring the error between the position of each vertebra in the model and the position of each corresponding vertebra in the measured subject data. The errors for the vertebrae positions are optionally weighted and then summed to produce an overall error value for the current pose 35 (e.g. standing). The procedure then returns to step 34 and a different pose (e.g. sitting) is selected and an error for that pose is determined at step 35 and so on. In one example, seven different poses are assessed. Once all of the poses have been assessed, the errors for the poses are then optionally weighted and summed to determine a total error for the set of poses in step 36.

The next step of the optimisation procedure is to determine a Jacobian matrix for the model 37. This is done by adjusting the parameters independently and in combination to determine their effect on the error. The Jacobian matrix describes the direction each parameter should be adjusted for the next iteration of steps 34-38. At step 38, the parameters of the model are adjusted based on the corresponding entries of the Jacobian. Parameters that can be adjusted include the spring coefficient, number or position of linear springs, a mass of or attached to a vertebra, and the damping factor of a damper. The model is then assessed with the newly adjusted parameters to determine a total error for the new parameter values in the next iteration of steps 34-38.

Steps 34-38 repeat until they converge on an optimised solution. This is determined in step 39. The model is then constructed using the optimised parameter values 40.

In the example just described, the parameters are all optimised together in a multiparameter optimisation procedure. This may be advantageous because the effects of the different parameters are typically not orthogonal to each other. However, in some cases it may be possible or suitable to optimise subsets of parameters independently from others, or even to optimise parameters individually.

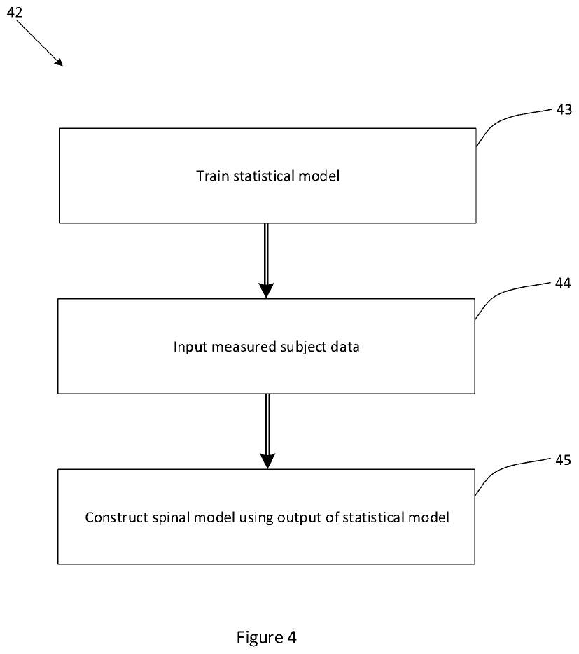

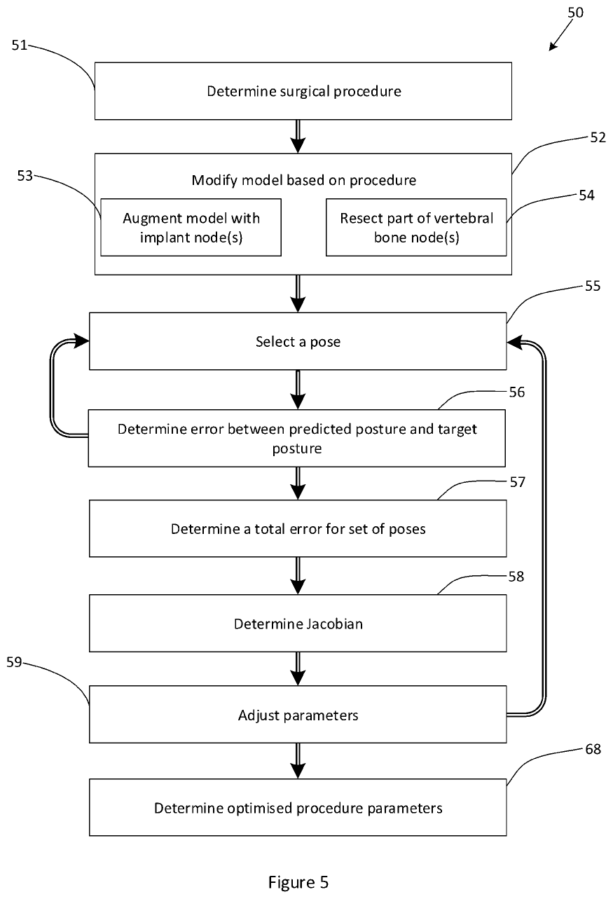

A statistical optimisation procedure 42 is shown in FIG. 4. A statistical model is initially trained 43 on a set of subject measurements (e.g. functional data measurements and imaged geometric data) and determined mechanical parameters. Measured patient data is input to the statistical model 44 to determine the most likely set of mechanical parameters given the input data. The input clinical data can be imaging data in one pose in combination with functional data. The functional data can be measured using wearable sensors as described previously. The model is then constructed 45 in accordance with the output of the statistical model.

The method of FIG. 4 can be used instead of the method of FIG. 3 or in conjunction with it. In one example, the method of FIG. 3 is performed on an initial set of subjects. The inputs and outputs of this method are used to train the statistical model of FIG. 4. Once the statistical model has been adequately trained, new subjects are modelled using the statistical model of FIG. 4 rather than the numerical optimisation of FIG. 3.

FIG. 5 is a flow chart showing a method 50 of determining the effect of a surgical procedure and optimising the surgical procedure based on a target posture for the subject. At step 51, it is determined what surgical procedure is to be performed. The surgical procedure could be spinal fusion, spondylolisthesis correction, scoliosis correction or another suitable procedure. Some procedures involve the implantation of devices into the subject's spine. For example, surgical procedures can involve implantation of interbody cages, pedicle screws, rods and/or tethers. Some procedures involve resection of part of a vertebra. At step 52, the model of the subject's spine is modified based on the determined procedure. This can involve augmenting the model with one or more node units representing one or more rigid surgical implants such as interbodies, and/or one or more spring units representing one or more flexible surgical implants such as rods, in step 53. This step is performed if the procedure includes use of one or more surgical implants. Modifying the model can also involve modifying one or more of the vertebral node units based on one or more resections associated with the procedure, if the procedure involves resection, in step 54. The virtual model adapts to the augmentation(s) and/or resection(s) to determine the effect that the procedure will have on the subject's spine. This may give a surgeon a better understanding of whether a planned surgery will achieve the desired clinical outcomes before the surgery is performed. This may lead to better patient outcomes and fewer unsuccessful surgeries.

The method 50 then enters a numeric optimisation loop of steps 55-59. At step 55, a pose is selected and the model is used to predict the subject's post-operative posture in at least one pose. The predicted posture for this pose is compared to a target post-operative posture (e.g. a desired segmental or global lordosis or kyphosis angle) and an error that reflects the difference between the modelled posture and the target posture is determined 56. This may be performed in a similar way to the error between the modelled posture and measured subject posture in step 35 of FIG. 3. In this example, the comparison is performed for a range of poses by reiterating steps 55 and 56 and a total error across the range of poses is determined at step 57.

A Jacobian matrix is then determined for the model (as modified based on the procedure) in step 58 and the parameters associated with the procedure are adjusted based on the Jacobian in step 59. Specifically, if the procedure includes one or more implants, parameters of the implant(s) are adjusted or the type of implant is changed. If the procedure includes one or more resections, parameters of the resection(s) are adjusted. Steps 55 to 59 then repeat until the implant and/or resection parameters converge on an optimal solution that best matches the target posture, as determined in step 68.

As with the method of FIG. 3, this may be a multiparameter optimisation procedure with all parameters optimised together but in some cases subsets or individual parameters may be optimised independently.

This method allows the parameters of the surgery to be optimised to best achieve the desired clinical outcomes. The optimisation method allows different combinations of implant types, parameters, numbers etc. to be assessed, individually or in combination. The adjustable implant parameters can include interbody size, shape, position, and orientation; screw diameter, length, position, orientation, depth, and tension; rod length, diameter, and shape; or other suitable parameters. The implants can be different types including interbodies, rods or screws or implants made by different manufacturers. Resections of different parameters (such as location, shape, size) can also be assessed, alone or in combination with other resections or in combination with various implants and combinations of implants.

In particular, the effect of one component can be compared to the effect of another component to see which will best achieve the target posture. The effects of various different parameter values of a component can be compared to optimise the parameter to achieve the target posture. The effect of one or more additional components can be assessed taking into account the effect of another component with fixed parameters (for example because it has already been delivered). The effect of one component of a set can be assessed taking into account the other components of the set. In this context, “component” refers to implants or resections.

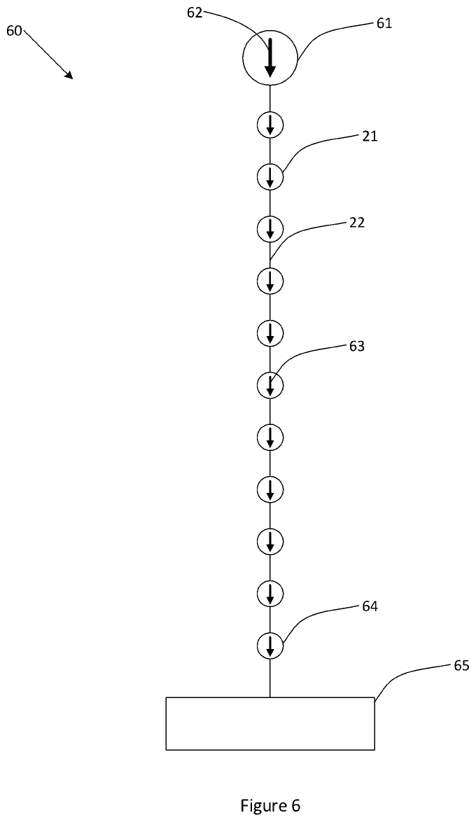

FIG. 6 is another exemplary spine model 60, shown in a coronal plane. The model includes node units 61 connected by connector units 62. In this model, the spine includes a superior node unit 63 that has a mass 64. Mass 64 represents the mass of the subject's head. The model 60 also has an inferior unit 66 that is constrained with respect to the base 67. The inferior unit 66 is constrained in 6 degrees of freedom, representing the fixed connection of the base of the subject's spine to their sacrum or pelvis. Each of the nodes can have a mass 65 representing the segmental load on the respective vertebra. The model can also include force sources representing muscle forces on the respective vertebrae.

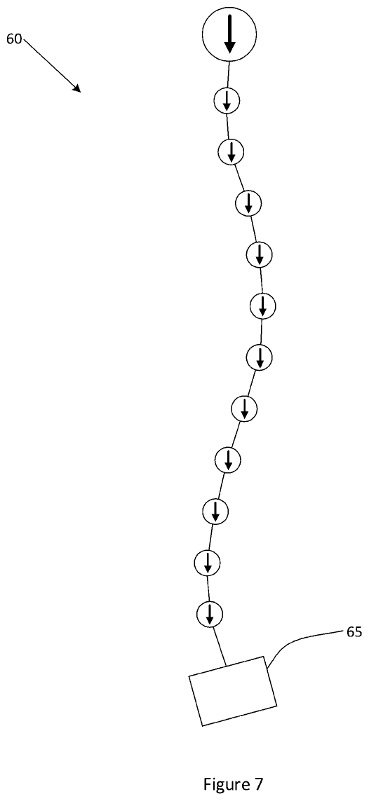

FIG. 7 shows the model 60 in the sagittal plane. It can be seen that the base 67 is tilted, corresponding to the orientation of the subject's sacrum and/or pelvis in the standing pose. The base 67 can be rotated into different orientations to simulate different poses.

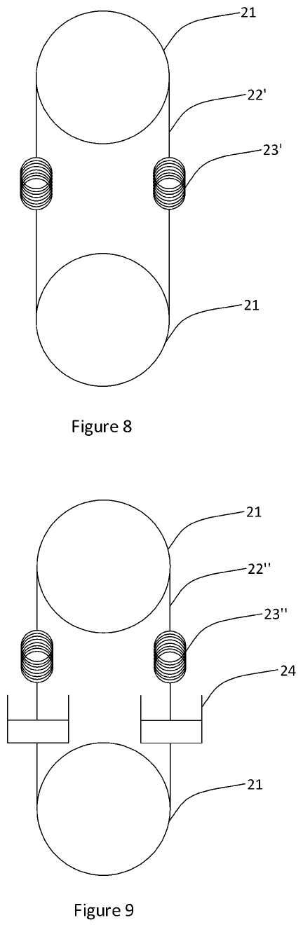

FIGS. 8 and 9 show alternative connector units. In FIG. 8, node units 21 are connected by connector unit 22′. Connector unit 22′ includes two linear springs 23′. The springs 23′ are spaced apart on the node units 21. The positions of the springs 23′ on the nodes, as well as their spring coefficients, are parameters that can be optimised when constructing the spinal model.

In FIG. 9, the node units 21 are connected by connector unit 22″. Connector unit 22″ includes linear springs 23″ and dampers 24. The dampers 24 can be dashpots or viscoelastic elements. The damping factors of the dampers 24 are also optimisable parameters.

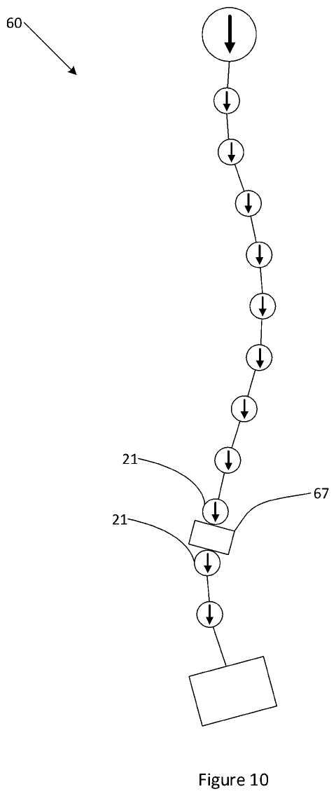

In FIG. 10, the spine model 60 has been modified to simulate the effect of a surgical procedure. Specifically, fusion implant 67 has been implanted between two node units 21. This changes the coupling between these two nodes, causing the rest of the spine model 60 to change as the forces on the spine are balanced differently. The effect of this procedure can be displayed on a user interface, for example graphically on a visual model of the spine or numerically in a list of values describing posture.

The methods and models discussed can be implemented by instructions on a computing device. These can constitute a computer program that is embodied in various media, including tangible or non-tangible media, transitory or non-transitory media and can run on any suitable device. There can also be provided instructions for outputting data to a display device and for producing a user interface (UI) on the computing device.

For example, when estimating the effect of a resection, the method can include calculating how much adjacent vertebrae will intersect with each other in a target pose or posture, highlighting in the UI the region of intersection, and suggesting the region of vertebra to cut away based on the intersection. The user can interact with the UI to resect the suggested region from the vertebra in the model and simulate the effect of this on the model.

The methods discussed can be performed pre-operatively, intra-operatively or post-operatively. Performing the methods pre-operatively helps select the best implant(s) and/or implant parameters to achieve the optimal posture. Performing the methods intra-operatively helps select the best implant(s) and/or implant parameters based on an already-delivered implant or already-planned parameters.

Performing the method intra-operatively on a set of already-delivered implants allows the user to update predicted post-operative function during the surgery if the surgeon decides to use different implants, resect the bone differently, or find additional constraints. It also gives the surgeon up-to-date information during the surgery.

When performing the method post-operatively on a set of already-delivered implants the methods can provide an updated post-operative function prediction. This can then be used by the surgeon to manage expectations for the patient and be used in physiotherapy as guidelines for expected function. The methods can also give the surgeon feedback on how their plan and execution may differ, and improve the model parameter estimation algorithms and the forward simulation algorithm by comparing actual to estimated post-operative posture.

The methods and programs described may allow simulation of the outcome of a surgical procedure without the need to perform it, improve accuracy of a simulation of a spine, reduce computational complexity of simulation of a spine, facilitate optimisation of a parameter of a surgical procedure, and facilitate selection of an appropriate implant or resection for a subject.

While the present invention has been illustrated by the description of the embodiments thereof, and while the embodiments have been described in detail, it is not the intention of the Applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the spirit or scope of the Applicant's general inventive concept.

What is claimed is:

1. A method of determining effect of a surgical procedure on a subject's spine, the method including:
receiving geometric data of the subject's spine;
constructing a virtual model of at least part of the subject's spine using the geometric data, the virtual model including a series of node units representing vertebrae and a plurality of connector units, each connector unit including one or more virtual springs configured to model coupling between adjacent node units; and
determining the effect of a surgical procedure on the subject's spine using the virtual model.

2. The method of claim 1, wherein one or more virtual springs is a torsion spring.

3. The method of claim 1, wherein one or more of the connector units includes a plurality of linear springs that are spatially offset from each other.

4. The method of claim 1, wherein one or more of the node units further includes or is coupled to a mass representative of a load on the respective node unit.

5. The method of claim 1, wherein one or more the of node units further includes a force source representative of a muscular force on the respective node unit.

6. The method of claim 1, wherein the series of node units includes an inferior node unit that is constrained with respect to a reference object representative of the subject's sacrum, pelvis or both sacrum and pelvis.

7. The method of claim 1, wherein the series of node units includes a superior node unit that includes or is coupled to a mass representative of the subject's head.

8. The method of claim 1, wherein one or more of the connector units further includes a damping element.

9. The method of claim 1, wherein the model further includes one or more additional connector units connected between non-adjacent node units, each additional connector unit including one or more virtual springs.

10. The method of claim 1, wherein constructing the virtual model includes performing numeric optimisation of the virtual model by adjusting one or more parameters of the virtual model to minimise a measure of a difference between a posture of the subject's spine and a modelled posture of the subject's spine determined from the virtual model.

11. The method of claim 1, wherein the surgical procedure includes installation of a surgical implant and determining the effect of the surgical procedure includes augmenting the virtual model with a node unit representing the surgical implant.

12. The method of claim 1, wherein the surgical procedure includes resection of a part of a vertebra and determining the effect of the surgical procedure includes modifying one of the node units based on geometric data of the resection.

13. The method of claim 1, further including:
receiving functional data relating to the functioning of the subject's spine;
wherein constructing the model further includes inputting the functional data to a statistical model relating functional and geometric data to spinal model mechanical parameters.

14. The method of claim 1, further including:
receiving disc data relating to the subject's intervertebral discs; and
wherein constructing the model further includes using the disc data.

15. The method of claim 1, further including:
performing optimisation of the surgical procedure by adjusting one or more parameters of the modelled surgical procedure to minimise a measure of a difference between a target posture of the patient's spine and a modelled posture of the subject's spine as it would be after the surgical procedure.

16. A non-transitory computer-readable storage medium storing instructions thereon, the instructions when executed by a computer cause the computer to:
upon receiving geometric data of the subject's spine, construct a virtual model of at least part of the subject's spine using the geometric data, the virtual model including a series of node units representing vertebrae and a plurality of connector units, each connector unit including one or more virtual springs configured to model coupling between adjacent node units; and
use the virtual model, determine the effect of a surgical procedure on the subject's spine.

17. A method of determining effect of a surgical procedure on a subject's spine, the method including:
receiving geometric data of the subject's spine;
constructing a virtual model of at least part of the subject's spine using the geometric data, the virtual model including a series of node units and a plurality of connector units, each connector unit including one or more virtual springs configured to model the coupling between adjacent node units, wherein coupling been adjacent node units represents an intervertebral disc; and
determining the effect of a surgical procedure on the subject's spine using the virtual model.

18. The method of claim 17, wherein one or more virtual springs is a torsion spring.

19. The method of claim 17, wherein one or more connector units includes a plurality of linear springs that are spatially offset from each other.

20. The method of claim 17, wherein at least one node unit represents a vertebra.
